(19) United States
(12) Patent Application Publication
(10) Pub. No.: US 2010/0112680 A1
(43) Pub. Date: May 6, 2010

(54) INDICATOR SYSTEM FOR DETERMINING ANALYTE CONCENTRATION

(76) Inventors: Paul Nigel Brockwell, Emerald Beach (AU); Robert Vincent Holland, North Ryde (AU)

Correspondence Address: THE WEBB LAW FIRM, P.C., 700 KOPPERS BUILDING, 436 SEVENTH AVENUE, PITTSBURGH, PA, 15219, US

(21) Appl. No.: 12/307,981

(22) PCT Filed: Jul. 11, 2007
(86) PCT No.: PCT/AU2007/000954

§ 371 Date: Dec. 8, 2009

(30) Foreign Application Priority Data
Jul. 11, 2006 (AU) 2006903719
Aug. 14, 2006 (AU) 2006904407
Feb. 28, 2007 (AU) 2007901030

(51) Int. Cl. C12M 1/34; G01J 1/48; G01N 21/00; G01N 27/26

(52) U.S. Cl. 435/287.9; 422/86; 422/55; 204/403.01; 204/409

(57) ABSTRACT

A method for quantitatively sensing, using an indicator system based on diffusion in space and time of a reaction front, for determining and reporting the prevailing concentration or exposure history of an analyte in food, beverage, and pharmaceutical monitoring for the state of quality, for ripeness indication in fruit, for monitoring environments for concentrations of sanitisers, pollutants and nutrients, for monitoring the residual life of filters, and for monitoring stream flows.

### FIELD OF THE INVENTION

The invention generally relates to devices and methods for sensing changes in the concentration of an analyte or exposure history of an analyte that participates in a chemical reaction that affects the control over quality in the fields of food beverage quality, pharmaceutical spoilage, personal protection and environmental integrity.

### BACKGROUND OF THE INVENTION

There are several gas detection technologies incorporated into electronic instruments that employ coloured indicators, usually combined with luminescence, fluorescence, reflectance technologies. These instruments require the manual operation, calibration, and interpretation of trained technicians. Examples of patents that include such instruments include GB2102947, U.S. Pat. No. 5,094,955, WO0077242, WO9627796, U.S. Pat. No. 6,908,746, which can be used to detect spoilage products from bacteria in food and blood, and U.S. Pat. No. 2,890,177, U.S. Pat. No. 3,068,073, U.S. Pat. No. 3,111,610, U.S. Pat. No. 3,754,867, which can be described as gas detectors.

Visual readings are used to interpret values in sample tubes manufactured by Draeger® and are used by technicians with suction pumping to extract gas samples and expose coloured indicators disposed in a sample tube to the target molecules to obtain a visual measurement by means of a moving coloured band. Similar technology, which manually samples extracted spoilage gas in food containers and reports the attainment of a predetermined threshold value as a PASS/FAIL test, is disclosed in U.S. Pat. No. 5,653,941.

It would be a useful technological contribution if such technologies could be incorporated into passive indicator systems, i.e. systems that do not require human intervention, that run under expert design to meter exposure and report values interpretable by non-expert audiences, not just by technicians. There would be several industrial applications for such passive indicator devices, such as for food quality (microbial spoilage), the surface of fruit as a freshness indicator, package integrity (including tamper-evidencing), human exposure to toxic gases, residual life of filter cartridges in gas masks, expired air from patients lungs, evaporation-condensation indicators, sample kits for urea in blood and urine.

Other indicators simulate real environments with analogue systems. Classic among these are the time-temperature indicators that report thermal exposure with reactants that share similar activation energy and rate constant as the system being thermally modelled, and the correlations drawn provide inference as to the condition of the real system (Riva, M. 1997).

Other indicators simulate real environments with analogue systems. Classic among these are the time-temperature indicators that report thermal exposure with reactants that share similar activation energy and rate constant as the system being thermally modelled, and the correlations drawn provide inference as to the condition of the real system. More recent indicators have been developed that meter exposure to an analyte directly responsible for changes in an environment. The metering, however is restricted to the attainment of a threshold value, and the communication, consequently, is limited to an ON/OFF or PASS/FAIL reading. Such an indicator is commercialised by Food Quality International for monitoring the quality of meats and fish, and by Ripesense for the ripeness of fruits. The limitation with these devices is that reliance is placed on a change in visible colour spectra to the observer, with reference to a colour chart to determine end-point. No numerical scale is obtainable for interpretation purposes with these devices, and the observer is left to judge colour spectra for the determination, which is problematic with resolution and accuracy.

No invention, however, has claimed application to include a measuring device that uses scavenging action to actively diffuse the target molecules of a chemical reaction responsible for quality changes, or markers associated with changes in the integrity of environments, through engineering structures in a direction that establishes a moving front, in synchrony with changes in the quality of an environment being studied. The present invention uses this moving reaction-front to create a sensor in an instrument that measures and reports either prevailing levels of target molecules (the analyte), or exposure history.

The reading provided by the novel device according to the present invention generates a point along a continuous numerical scale, with no upper limit, and consequently, caters for the demands for hard data in quality assurance for today's medical industry.

Whereas the prevailing level of the analyte provides information as to the acceptability of the analyte's concentration in the environment, the reported cumulative exposure is intended to result from the additive accumulations of reactions that occur with the analyte at various, times during the deployment of the device.

Such an instrument, now disclosed, can be deployed in the confines of any closed or partially confined or steady-state condition of a real-environment containing the target molecules, or in a sample stream flowing into or out of such environment, gaseous or liquid, through which target molecules pass. Typical environments of interest to the present invention include biological spoilage reactant or product in food or biological products, environmental pollutant, or treatment product or pesticide for the sanitisation of air or water and the integrity of gas-seals in packages.

### SUMMARY OF THE INVENTION

It is therefore a general object of the invention to provide a chemical exposure history of a closed or partially closed real-environment by reporting contact with, or release of, target molecules in relation to that environment.

Accordingly, in one aspect the invention relates to a method of monitoring the chemical exposure history of a closed real-environment by reporting the contact with or release of target molecules in relation to that environment, comprising the steps of;
locating a monitoring device within the confines of the closed real-environment, or in a sample stream through which the target molecules pass, into or out of said environment, wherein said monitoring device has a permeable substrate, and records exposure to target molecules by measuring diffusion of those molecules through said substrate; then,
periodically, during the exposure period and/or at the end of the exposure period, recording the degree of molecular diffusion of the target molecules through the substrate;
   so as to provide an exposure history of the environment in relation to the contact with, or release of, target molecules.

The target molecules may be molecules of interest to quality management and may include: biological spoilage reactants or products, pollutants, or sanitising chemicals to treat air or to treat water to improve quality. The target molecules of interest may be associated with food spoilage, biological product spoilage, microbial and chemical degradation, personal protective equipment, environmental conservation and other environmental monitoring applications.

Suitably, the permeable substrate of the monitoring device has one or more chemical indicators disposed therewith which indicate the diffusion of a target molecule into the substrate

Suitably, the target molecule induces a chemical transformation in the substrate such that the presence of the target molecule within the substrate is indicated. The chemical transformation may be an oxidation—reduction reaction or may an ionisation reaction such as induced by a change in pH. The chemical indicator may therefore be a pH indicator.

The chemico-physical properties of the permeable substrate, such as density and porosity, and/or size of aperture of the intake into the substrate, may be varied to increase or decrease the rate of diffusion of a target molecule through the substrate.

Suitably, the degree of diffusion of the target molecule through the substrate is metered by reaction of the target molecule with the chemical indicator.

In some embodiments, the degree of diffusion reports concentration of the target molecule in a continuous scale of moving linear colour band or moving colour ring.

Suitably, the monitoring device comprises a chamber wherein the substrate is disposed in the chamber, said chamber configured to ensure that the rate of colour change with distance in a continuous scale is achieved by ensuring that the reaction time at the front of the migration proceeds, in step with, the diffusion of the target molecule in the substrate.

The monitoring device may report the prevailing level of a target molecule or cumulative exposure to a target molecule, or as an integrated device it may report both the prevailing level and exposure history.

The monitoring device may be comprised of a reaction front, which is commensurate with the degree of diffusion of the target molecule within the substrate of the indicator device.

The indicating device may confine the indicator reaction front along a continuous scale by disposing the indicator medium in a narrow and elongated tube to confine the diffusion along the indicator in a progression along a plane to the observer,

The monitoring device may confine the indicator reaction front along a continuous scale by disposing the substrate in 2-dimensional form as a thin layered disc or of variable thickness, with impermeable upper and lower surface, to confine the diffusion in a progression migrating from the outer edge to the inner centre to the observer, or alternatively, from the centre to the outer edge.

Suitably, the substrate is disposed in a 2-dimensional form such as a triangular shape or alternatively in a 3-dimensional form as wedge, cone or pyramidal form, or other tapered form or other form of variable thickness.

The monitoring device may be made to diffuse further along an increasing non-linear scale by varying the thickness of the substrate which comprises the indicator, along the length of a linear strip as in the case of the thermometer form of the invention to create a wedge; or increasing the thickness along the radian of an arc of a circle present in the disc form of the invention to create a hemispherical or hemiovular shape in the case of the disc form of the invention. By making the intake end the tapered one, progressive diffusion becomes more non-linear with increasing distance of migration. Alternatively, the diffusion can be made more linear by diffusing from a thick end of the device to a thin one.

The monitoring device may report the concentration of a target molecule in a discrete scale by deployment of masking coloured print in stations over the moving colour band so that the arrival of the band at a station is observed by a colour change at the station, or where the colour of the band itself masks the appearance of a print below, and the progressive migration of the colour band alerts the observer to the attainment of new levels of exposure by colour loss in the previously masking band and appearance of the message below,

The monitoring device may report cumulative exposure to a target molecule such as carbon dioxide by the use of reactants within the substrate that produce semi-stable reaction products—reversible with mild heating in the range 50-80° C., or with stable reaction products—reversible only at oven temperatures.

Suitably, the monitoring device reports the prevailing level of a target molecule through reactants—including buffers, deployed with the substrate, that produce unstable reaction products at ambient temperatures making the reaction immediately reversible, so as to generate reports of prevailing levels of analytes.

The monitoring device may report either prevailing level or cumulative exposure in a readable scale whether by visual colour movement or separation in space possibly measured as the quantum of reflected light within a field of view of an instrument, or as colour spectrum or colour intensity, or with the aid of an instrument that measures colour development as wave length or frequency, reflectance, luminescence or fluorescence or other radiative technology, such as a bar-code scanner at a supermarket.

The monitoring device may report either prevailing level of cumulative exposure by changes in an electrical signal attached to a digital display or transponded by radiative technology to a coordination centre and possibly relayed internationally by internet or satellite communications.

The monitoring device is comprised of colouring agents with the indicator substrate, or it may use masking or background layers of colour in order to alter the colour or legibility of the substrate as seen by the observer or by the reading obtained with an electronic scanning instrument.

The mode of communication to target different audiences, with respect to the monitoring device, may be varied in coded communications interpretable by only a targeted recipient class of people, to communicate the exposure of the device to the target molecules.

The monitoring device may be calibrated by: selection of an appropriate chemical reagent to indicate for the presence of a particular target molecule, the concentration of reagent; or rate of diffusion into an indicating medium by varying the permeability of the substrate.

The permeable substrate of the monitoring device may be disposed in micro-spheres in a linear configuration in a tube in order to establish a degree of tortuosity and thereby slow diffusion to ensure that the reaction time at the front proceeds at the diffusion rate, and to calibrate the rate of migration.

The monitoring device may measure cumulative exposure by mixing an indicator reagent with a scavenging reagent.

In some embodiments, the monitoring device may be mounted as an adhesive label or tag in thermal contact with a package or vessel containing a food or biological product.

Suitably, the monitoring device may be deployed as a stand-alone instrument for insertion into packages; as an adhesive label or print for deployment on the internal wall of packages, as a laminate protected with solvent-proof material, or on the external wall of permeable packages.

A protective filtering layer may be disposed over the monitoring device, or within close proximity, to scavenge non-target molecules from the environment being measured and so provide selectivity in the measurement as to target molecules and render the monitoring device solvent-proof.

Preferably, the monitoring device is used to monitor food, and environmental quality applications, and applications that monitor the growth of cultures of microorganisms.

### BRIEF DESCRIPTION OF THE DRAWINGS

The invention will now be described on the basis of non-limiting examples shown in the drawings:

FIG. 1: illustrates an indicator wherein the indicator gel is disposed linearly and is covered by a barrier layer to confine diffusion in one dimension;

FIG. 2: illustrates a section of a linear indicator device;

FIG. 3; illustrates an indicator device in the form of a dip-stick instrument for submergence in liquids;

FIG. 4: illustrates planar diffusion in two dimensions from the edge of a film toward the centre;

FIG. 5: illustrates an aerial view of a disc form of an indicator that applies planar migration during operation;

FIG. 6 illustrates an indicator device in a tapered form such as a wedge, pyramid, cone or other tapered shape, so that colour change will progress with increasing exposure from the fine tip to the thick base;

FIG. 7 illustrates a moving colour band migrating from left to right;

FIG. 8 illustrates a monitoring device applied to fruit;

FIG. 9 illustrates a monitoring device inserted into soil; and

FIG. 10 illustrates a monitoring device mounted in the exhaust stream of a motor vehicle.

### DETAILED DESCRIPTION OF THE INVENTION

Two types of measurement are possible in the present invention: the prevailing level and cumulative exposure. The first measures the level of an analyte recorded at the time of measurement, whilst the second meters accumulated units of exposure in an additive manner and reports the history of exposure. In both cases of exposure, the metering and reporting can be along either a discrete and graduated scale, or along a continuous scale, resulting from the moving band of a reaction front. Readings may be visual or electronic. The observation may be targeted at the unskilled, as with visual readings, or to those skilled in the use of instruments and be reported to a remote control centre as with electronic readings transponded using radio waves or by other electromagnetic means.

Food, and biological preparations lose quality during distribution when they are exposed to heat for some time and when they are contaminated with spoilage organisms. Quality loss and residual quality can be measured with the products of metabolism from bacteria and fungi that spoil food. Example analytes include carbon dioxide, hydrogen sulphide, sulphur dioxide, hydrogen and ammonia gases, acetic and lactic acid, ketones and aldehydes. Chemical breakdown under refrigerated storage of foods like meat and fish, can be measured by the formation of amines from degrading proteins. The formation of limonin, a bitterness product in degrading orange juice can be similarly metered. Loss of quality in packaged food can also be measured by oxygen influx and consumption in prepared foods, and by declining concentrations of oxygen in packaged produce due to anaerobis resulting from respiring plant tissues being held at temperatures that exceed the design limit of the food packaging.

The breakdown products of respiration, spoilage activity and chemical degradation are often acids, bases or oxidation-reduction products, whilst the reactants typically include oxygen. Monitoring the formation of breakdown products, or the utilization of reaction products, can indicate the progress with biochemical and chemical processing.

pH or oxidation/reduction indicators can be used to monitor spoilage in the confines of packages or within diffusing gaseous or liquid streams undergoing environmental changes, downstream or upstream of the site of activity. Such indicators can be disposed in a package environment, other confined space, or within a sample stream in proximity of the site of generation, to monitor levels of exposure.

The indicator can react with the acid or base evolution product and meter the progress of a titration reaction as kinetic exposure by the formation of conjugate acids/bases using a pH indicator with or without pH buffer. Similarly, with oxidation-reduction reactions, indicators can be used to meter progress with exposure over time to varying concentrations of analytes, such as oxygen. Condensation and evaporation indicators can be similarly deployed as meters for moisture migration into packages of food.

Many foods, for example milk, are safe at low bacterial populations. The issue for the consuming public is quality and the acceptable limit may vary between individual consumers. Cumulative heat-exposure will permit populations of spoilage organisms to develop. Milk and like products are marketable up to a point, reporting mere presence or absence of bacteria is of little value. In these cases it is valuable to meter the population and its metabolism with measurement of accumulated carbon dioxide evolution or other spoilage product, such as spoilage acids. The problem with prior art, where indicator films changed colour, is that it merely reported the attainment of some threshold value, or relied on an instrumental reading of colour intensity. The improvement in the present invention is to report readings in a lateral spread, enlarging with increasing exposure like a conventional colour-band thermometer.

Fresh produce such as poultry eggs, fruits, vegetable and flowers respire at an Arhennius rate with temperature, and the respiration can be modified by various atmospheres of oxygen and carbon dioxide. Prevailing levels of oxygen and carbon dioxide levels, measured at the surface of the epidermal cells as a fruit-sticker, or as an internal sticker on the wall of a package, reflect the prevailing environmental conditions of temperature and gaseous atmosphere, whether conserving or abusive of the postharvest life of the produce, Similarly, a sticker can be placed against the exterior surface of the shells of poultry eggs to meter either the respiration of the egg, the spoilage products of bacteria inside the shell or both.

With fresh produce, the accumulated respiration of a mass of cells can be used to meter freshness as ‘respiration-life’ of various severed plant organs (Brash et al. 1995, Bower, J. H. 2001).

Ethylene levels, prevailing and cumulative, can herald the onset of ripening in climacteric fruit or indicate a stage of ripening when fruit such as pear, avocado and kiwifruit is optimally ready-to-eat, without the need for pressure testing with fingers and damage to the fruit.

Permeable covering layers are present in the epidermis of produce organs. Oxygen, carbon dioxide, ethylene and alcohols in plants permeate these surfaces and present an opportunity for measurement of equilibrium levels with the present invention.

Evolved carbon dioxide, ethylene and other gases such as ethanol from the cells of produce, and passed by diffusion through the layers of epidermal cells to the surface, can be scavenged by the indicator device of the present invention, into an overlying sticker mounted onto the produce itself, or through the walls of permeable packaging used in the trade to market produce into a sticker mounted on the outside of packaging. Alternatively, the device may be incorporated as a layer within the packaging material, or be deployed as an independent device into a package, water-proofed and leakage-proofed, or on the outside of non-transparent packages with connection tubing.

In the case of food, health products and other perishable products or medical specimens, in other non-permeable containers, such as transparent glass jars, attachment of a solvent-proof label to an interior wall enables metering and reporting functions to occur. Should a non-transparent container be used, a pin-hole may be punched into the vessel of for example, polyethylene or other polymer, and the label-device can then be applied as a sealing-patch in the same manner that a puncture in a bicycle tube is repaired.

Alternatively, a bayonet fitting through a pin-hole punched in the package wall and connected with a tube to the intake of a metering tag may be used to deploy the metering device. These methods enable monitoring to be undertaken in non-transparent vessels and containers.

The definition of ‘packages’ may extend to the outer-packages of several smaller packages and may include large containers, including shipping containers. Measures obtainable include the current state of respiration and ripening, or the respiration or ripening history of the produce.

For effective quality management during distribution, in the modern audit trails wherein transparency and accountability in dealings is sought, it is desirable to report on the progressive deterioration in products from harvest or food processing until the point of eventual consumption. Hence a metering system is desirable to show the degree of expiry in the product's life whilst in the hands of each party in transport and storage.

The quality of fresh produce deteriorates with delay in handling and sub-optimal temperature management during distribution, freshness is lost. Freshness in the trade is greatest when a fruit is picked, or ripening is commenced. Despite the conservation provided by freezing, canning or other methods of food preservation to processed foods and beverages, contamination by spoilage organisms and chemical degradation will eventually limit storage and shelf-life. Monitoring the state of freshness is a challenge addressed by the present invention.

The quality of food deteriorates with thermal exposure during distribution, as contaminating microorganisms grow and multiply. The metabolism of microorganisms is a principal factor in degradation of food, and is regulated by factors including temperature, gaseous oxygen and carbon dioxide concentrations, growth media, water activity, inhibitors to growth and preservatives. Temperature-time indicators, therefore do not reflect the totality of environmental factors regulating microbial growth, particularly with the formulation of mixed-foods, therefore monitoring changes in the real system will be more accurate for quality control than predictions in the simulated one.

This chain of distribution often involves the cooperation of many disparate parties and exposure to heating and delay between harvest or food processing and household consumption. Freshness is greatest when manufactured food is packed. Modern distribution systems involve passage from one link in the distribution chain to the next, commonly including: manufacturer inventory for processed food, and harvest-cooling and packing-storage at the packing-house for fresh produce. Distribution then commonly involves road, rail, sea or air transport followed by wholesale inventory-retail inventory-retail display-customer purchase-customer storage.

During distribution throughout the market chain, various parties are interested in the quality of produce and food and this would be beneficially reported on the surface of individual fruit or food package by a communication device. This information can represent marketing intelligence and one party, for example a retailer, may wish to obtain early warning on the quality of a food product for internal quality management purposes, before the information is passed onto the consumer-customer. This would allow the retailer to intervene and either remove the product from sale, or to discount it for a quick sale.

To protect their reputation in the market for good quality products, retailers prefer to restrict the information available to customers about food quality whilst ensuring the safety of their food products with internal management systems, behind the scenes, without alarming customers to imagined or perceived risks. Similarly, they may elect to reject consignments from wholesalers as not fit for purchase. In order to target the communication of the quality status to various audiences, it is desirable to use coded signals.

The present invention satisfies this need by communicating the rate of colour change in an indicator with distance using the migration of a colour band. It thereby effects a greater reliability in reporting the population of decaying organisms and their activity and the metabolism of produce cells. The invention provides that communications on the status with quality are directed to respective parties along the distribution chain, commensurate with level of deterioration and liberation of spoilage products and/or the consumption of reactants. Such reporting according to the need-to-know is compatible with the realities of marketing and distribution.

An example of such coded messaging is to first deploy electronic detection of change in an indicator commensurate with early quality of loss by, for example, bar-code scanning by stock clerks or check-out operators at point-of-sale. At a more advanced stage, visual messaging could be combined with the bar-code and extend to customers post-sale if quality deteriorates further during customer handling. In the case of the householder as customer of the product, food can deteriorate to a greater level in a hot car on the way home from the shop and from poor temperature management during storage in the refrigerator and kitchen. The warning over food quality for this last party in distribution (the end-user), when deterioration is so advanced as to warrant the wastage of the food, may be better communicated in a visual form such as alarming symbol and text, widely interpretable and for all to see.

There are indicator systems in prior art that infer the conditions or the degree of thermal exposure, in a freeze-thaw episode and the like. The time-temperature devices are placed in thermal contact with food and biological products, like bagged blood, and share the same thermal history as the product being distributed. The enzymatic process of biochemical processing or physical diffusion process in these devices involve processes different to that being of the real system simulated, and are modelled and calibrated with the real system according to a correlation relationship.

There are devices in prior art that load respiring microorganisms with growth medium to produce acid products from respiration in response to thermal exposure, for example yeast that grows when frozen food thaws. However, no prior art deploy cultures of the very species being studied in the real system. In the case of milk and fish spoilage, it has become known in recent years that special bacterial species, the psychotrophs that grow at refrigeration temperatures, are primarily responsible for food spoilage in modern food distribution systems.

The present invention should more closely and accurately simulate the real spoilage process. An independent device, such as an adhesive strip on the outside wall of the food container, can be inoculated with cultures of the particular spoilage organism known to be responsible for spoilage. The micoorganism can be mixed in a chamber that opens into the intake of the sensor with a growth medium comprising a small sample of a formulation close to the real food, for example in dried, frozen or vacuum packed form, with levels of microbial contamination reflective of the real system, possibly dehydrated, and commissioning the device at the beginning of food distribution with hydration, ventilation from a vacuum-packed state, or moving from cold storage temperature to the ambient under distribution so that the organisms can grow and multiply.

According to this method, milk spoilage would be reported by a moving colour-band indication emanating from a small sample of re-hydrated culture of psychotrophic bacteria in dried milk, typical of the contamination level in normal processing wherein the sample is connected through tubing into an adhesive strip and the device is mounted on the outside of the milk container and in thermal contact with the food milk contents during distribution and household storage.

A similar application of the present invention is to monitor vacuum-packed food for the loss of seal within the package, as oxygen will influx if the seal is lost and growth of the inactive microorganisms, known to be aerobic and harmless in classification, will be triggered and colour will change in the indicator-meter in response to their growth and metabolism. In this case the device can be placed within the sealed package.

There are oxygen indicators for reporting food quality that report elapsed time of exposure to air (21% oxygen), as exposure timers, by exposing the indicator to the air surrounding the food package when a package is opened for use. The time that a package is left open can be thus related to anticipated exposure to micoorganisms floating in the air, as the exclusion effect of package seal is lost. Additionally, some crude correlation can be made against the anticipated oxidation of the food when the package is opened to the air by consumers.

However, the quality of food deteriorates during distribution to the consumer and it is desirable for food manufacturers and distributors to measure the exposure to the variable number of molecules of oxygen permeating a packaging material designed to be vacuum-sealed, or impervious to gas exchange, or that infiltrating pore spaces resulting from a break in a package's seal during storage, transport and marketing. This would provide a more accurate measure of the degree of oxidation in food itself during distribution. Further, measurement of the internal oxygen concentration of special packages permeable to respiring produce such as minimally processed vegetables is valuable to report progressive anaerobis, which not only causes rapid senescence of plant tissues but encourages the growth of dangerous anaerobic bacteria that threaten human health.

To achieve such measurements is an objective of the present invention with deployment of adhesive labels onto permeable package walls, composition of transparent package walls, and package inserts, for example tags placed into food packages to measure and report oxygen permeation through a barrier film, such as into a plastic bag of wine held in the bag-in-box package the wine ‘cask’, or through a bottle's seal.

Package integrity is important in food quality and safety, bacterial cells and fungal spores can enter through gaps in the package walls. Food packages lose their seal when they are damaged. Manufacturing defect also may fail to create an effective seal. Many packages are designed to achieve a seal against entry of bacterial cells in the air, but are not gas-tight for example some plastic milk containers. In these cases, the efficacy of a spoilage reporter is limited unless it can scavenge escaping gases or liquids, the products of spoilage, as they are produced. These gases or liquids, whether acid or alkaline in reaction, or the products of oxidation/reduction reactions, should be reacted with an indicator in a reaction which is semi-stable, otherwise a false reliance is placed on the reporting technology. Whereas prior art reported merely the attainment of a threshold level of acid/base, or oxidation/reduction product, this improvement scavenges and meters evolved reaction products in packages with minor leaks or design pores, that otherwise may have evacuated the package without detection.

A similar application is reporting the tampering of packaged products. Tampering with the packaging of food, pharmaceutical products and the like is preferably detected prior to sale electronically with a scanning device and only reported to customers if the scanning system fails to detect recent tampering. There are several indicators published in prior ark for reporting the loss of integrity in a package environment, some involving oxygen and carbon dioxide indicators. Food distributors, especially retailers, wish to achieve early intervention in cases of problems with package integrity, yet are obliged to warn the consuming public against health risks if their internal control systems fail them.

For improved industrial application, early detection is best reported with an early warning system, such as a disappearing bar code to retailers, whilst advanced detection from higher levels of reaction with indicators, is reported to customers with a printed message or symbol. The early detection can be achieved at a lower end of a discrete scale established by the metering system of the present invention, whilst the advanced warning is set at higher levels of exposure; although the communication modes differ, they reflect varying levels along a discrete scale.

Environmental monitoring of airs and waters for target molecules, including pollutants, is another application where the present invention can be deployed to monitor exposure to target molecules as a passive monitoring device.

The prevailing level within the environment is of interest, particularly when in sufficient concentration to cause alarm, such as carbon monoxide exhaust contaminating passenger cabins in motor vehicles, for this might risk acute poisoning; but also of interest is cumulative exposure from lower, insidious levels that may cause chronic poisoning, as in the case of unflued combustion room-heaters used in schools, or heavy metal ions in wastewater.

In the case of automobile emissions, cumulative exposure to a sampling device placed in the exhaust stream could report polluting cars, or meter emissions for the purpose of licensing, to permit access to inner precincts of polluted cities only to compliant vehicles, or vehicles within their license-to-pollute quota.

When monitoring the output of a chemical process, such as with pollution discharged from a vent or pipe, levels can vary over time, and reliance placed upon sampling at discrete points in time can lead to inaccuracies if concentrations over time are variable and episodic. Repeated measurements of the prevailing level to obtain a history of exposure are labour intensive and expensive. Continuous exposure can be a more reliable measure of the effect of chemical products in the environment. The present invention of an exposure indicating meter is innovative in providing this need. A detached sensor for remote deployment in a sample stream such as a chimney stack, a waste-water channel, or atmosphere such as ozone over a land mass from deployment with meterological balloons enables multiple monitoring stations to be monitored around the clock in an automated system, similar to data-logging. At the end of the monitoring period, the technician can obtain a visual reading or radio communication of the cumulative exposure, interpreted against the scale provided. The lower cost of manufacture in relation to electronic data loggers enables a greater sampling effort with more monitoring stations, and if by some adversity the inexpensive device is lost, then the repercussions are less severe to research budgets.

Fumigation and sanitation applications would also benefit from a monitoring technology that report levels of analytes in a scale. Water treatment, for example chlorination or oxidizing treatment of drinking water, swimming pools, sterilization of baby nappies, and the fumigation of rooms, produce packages, soils, also require information on exposure. The dosage is typically determined by calculation of the concentration of the analyte multiplied by time. Prevailing exposure levels and exposure history would be beneficially reported with the present invention by deployment of the sensing indicator device at a representative sampling point within the environment.

The problem with establishing a test vessel environment has been addressed above with deployment within package environments, the confines of a room in a building, measuring sample streams, passage through the wall of a permeable or porous plastic food bag, or a within a pollution vent or pipe. Attachment with tubing into the conductive vessels in plants can preclude the need to establish a sampling chamber, as can the use of tubing in connection with device into the generator of analytes such as an exhaust pipe, as can disposition within a protective yet permeable capsule for passage with the flow of liquids through piping. The device can be used in connection with tubing and other apparatus typically used in scientific instrumentation to obtain exposure to target molecules and obtain sampling streams.

The passive monitoring device of the present invention can be used to monitor microbial spoilage and chemical degradation in perishable products such as packaged food products.

The device may be made to selectively Meter exposure to those microorganisms that grow on packaged food and threaten human health, by bringing the indicator into direct contact with the food or biological product, or into a contact with a sample of the food or biological product in a separate chamber in thermal contact with the real environment of the food or biological product, and binding onto the indicator a known antibody to the targeted disease organism, or using certain indicators known to respond selectively to particular enzymes of spoilage bacteria or making indicators with a composition of antigen-sensitive molecules, or by use of selective antibiotics, fungicides or other growth inhibitors with specific action against contaminating species of microorganisms not being targeted for monitoring, but harmless for the species being targeted for monitoring.

It may be used to report oxygen and moisture migration into food packages, which cause deterioration in food quality. The device may be deployed as a laminate within the walls of packages, as a solvent-proof and non-leaching device for insertion with package contents, or as an adhesive label against the permeable walls of such packages.

It may be used to monitor the freshness of produce: fruits, vegetables, cut-flowers and foliage. It may report current levels of carbon dioxide, oxygen, ethylene, alcohol and other vapours of interest to homeostasis and senescence of plant tissues, as well as exposure history. With this information current state of homeostasis, senescence, freshness or state of ripeness may be inferred as well as residual life as a stored, transported and marketed product. The environmental conditions of atmospheric oxygen and carbon dioxide can also be monitored. It may be deployed as a laminate within the walls of produce packages, as a solvent-proof and non-leaching and safe-if-swallowed device (due to material selection for composition) for insertion with package contents, or as an adhesive label against the permeable walls of such packages.

It may be used to monitor plant health and homeostasis in intact plants by connection with injection apparatus into the relevant conductive vessels for water, nutrients or plant foods and enzymes; or by disposing the device as an adhesive patch onto the epidermis of the plant tissues being monitored to scavenge evolved gases.

The device may be used to monitor fermentation processing in food processing and manufacture, wine making and the composting of organic wastes and potting mixes. Similarly it can be used to monitor biological activity in soils.

The device may be used to monitor the prevailing level of a fumigant in the atmosphere of packaged food like grapes, or within a fumigated room, or under a fumigation blanket placed over soil or timber and the like, as well as the exposure history.

It may be used as a monitoring device to ensure effective dosing during water treatment with sanitising agents such as in the case of chlorination and oxidation of waters in swimming pools, and waters from dubious sources for potable use.

The device may be used to monitor the prevailing level and exposure history of a pollutant in airs, such as carbon dioxide, commonly used as an indicating gas for the range of polluting gases from the burning of wood and fossil fuels in buildings such as homes and school rooms. Accumulation of an undesirable gas in a relatively confined space such as the cabin of a motor vehicle may be reported, for example carbon dioxide causing drowsiness. Decisions concerning the need to ventilate occupied vehicle cabins and buildings may be supported by the information generated by the device.

It may be used to monitor the prevailing level and exposure history of a pollutant in waters, such as discharges from effluent pipes through channels into waterways, and may be fitted with string and flotation or weights, to dispose it at required depths of sampling.

The device may be used to monitor prevailing level and exposure history in a confined space for persons working with toxic gases, such as emergency workers, pesticide users, coal miners and spray painters, and may be disposed in the larger chamber of the workplace, or in the filtering cartridges of respirators worn by workers as personal protective equipment.

It may be used to monitor, by inference, the flow of air or water streams containing known concentrations of molecules targeted to generate an indication of exposure history, such as the ambient oxygen (21%) or carbon dioxide (0.04%) in air. An exposure model, with variables concentration, flow and time, can be adapted to calibrate the sensor to meter the volume of gas or liquid passing a sampling point in time, as a flow-meter.

One application of this method is to use the assumption model disclosed above for monitoring and replacing filtering devices in air or water streams, such as the air filters of combustion engines working in dusty environments, like agricultural tractors, or vacuum cleaners and air conditioners used domestically in the cleaning industry. Current industrial practice is to change or clean filters after so many hours of working-life, which assumes constant fan-speed. The metering sensor can be deployed to monitor exposure resulting from the variable fan-speed and air intake associated with episodal engine revolutions for engines at work. A related application is metering and heralding the need to clean swimming-pool filters when volumes of water have passed the sampling point of water flow. The improved simulation of the working-life of engines may serve as an improved measure over the current measures of engine-hours or odometer readings for vehicle travel. The cumulative oxygen intake or the cumulative exhaust, such as carbon dioxide, can more accurately represent the working-life and thereby the residual life of an engine, and be used to invoke servicing requirements and engine replacement needs.

The device may be used to monitor prevailing levels and exposure history of specific ions, including hydrogen (H⁺), in waters, airs, medical and veterinary specimens and plant sap.

It may be used as an indicator of moisture migration into packages and other spaces where it is desirable that conditions remain dry, by composing an indicator from known moisture absorbers and condensation indicators.

The monitoring device is typically comprised of an inert carrier medium, which may be composed of an inert water soluble carbonaceous polymer such as polyvinylalcohol. In order to ensure an aqueous chemical reaction, the carbon polymer may be polyvinylalcohol, polyvinylpyrrolidone or some other water-soluble polymer, or other transparent or translucent packaging material used in food and biological product distribution.

Plasticisers to establish a required permeation rate though the carrier medium may include propylene glycol, tetra methylene glycol, penta-methylene glycol or any glycol or polyhydroxyl material.

Exemplary pH indicators for reporting acid vapour presence or absence as colour change may be phenolphthalein, universal indicator, or other indicators changing colour around pH 8.0-10.0 range, or any other pH indicator, or combinations of different indicators to widen the colour possibilities or combinations of different indicators to widen the colour possibilities; and may be first dissolved in alcohol, or an appropriate polymeric solution.

The alkaline scavenging material may be potassium carbonate, sodium carbonate, calcium carbonate, or other carbonate of a strong organic or inorganic cation or an hydroxides or oxide of other strong organic or inorganic cations that is water-soluble; or any alkaline material. Examples include carbonates, hydroxides, or oxides of alkali metals or strong organic bases, which undergo a neutralisation process with acid vapours.

The acidic scavenging material may be acetic, tartaric acid, citric acid, and other weak organic acids.

pH buffers may be a carbonate or phosphate based one, an amino acid to undergo carbo-amino reaction, or any buffer to resist pH change.

Reagents that indicate the presence of ethylene include potassium permanganate, (colour change from purple to colourless or brown) and tetrazine derivatives (colour change from violet to colourless).

Reagents that indicate the presence of oxygen include leucomethylene blue, which can be considered a classic example for scavenging and indicating, together with many other leucodyes. The ones most similar to leucoMB [leuco thionine dyes] are generally colourless and oxidised to blue, green or violet dyes in the presence of oxygen. Another indicator dye is rubrene, bright orange in colour, which becomes colourless in the presence of both light and oxygen.

Barrier films to impede gaseous migration into indicator below may be composed of thin permeable plastic films such as polyolefins or polyvinylchloride.

Examples of water-proofing material and material that stop migration of reagents from the indicator device to food, whilst permitting gases such as carbon dioxide to permeate quickly include silanes like silicone.

Selective permeation of the target molecules such as carbon dioxide can be achieved by coating the carrier medium of the indicator with an encasing material like silicone or polyethylene.

Examples of suitable indicators, polymers and other appropriate reactive chemistries are disclosed in WO9209870 and extract is made of these disclosures.

“A large number of reactions are associated with colour changes. In each type of colour changing reaction there are several classes of compounds and each such class has several compounds which undergo a colour change. Below are some type of reactions and classes of compounds, which can be used as indicators and activators in the invention device.

Colour changing reactions and indicators are used for detection and monitoring of organic, inorganic and organometallic compounds. Such colour changing reactions and compounds are listed in a large number of books, reviews and publications, including those listed in the following references: Justus G. Kirchner, “Detection of colourless compounds”, Thin Layer Chromatography, John Wiley & Sons, New York, 1976; E. Jungreis and L. Ben. Dor., “Organic Spot Test Analysis”, Comprehensive Analytical Chemistry, Vol, X, 1980; B. S. Furniss, A. J. Hannaford, V, Rogers, P. W. Smith and A. R. Tatchell, Vogel's Textbook of Practical Organic Chemistry, Longman London and New York, p. 1063-1087, 1986; Nicholas D. Cheronis, Techniques of Organic Chemistry, Micro and Semimicrn Methods, Interscience Publishers, Inc. New York, 1954, Vol. VI, p. 447-478; Henry Freiser, Treatise on Analytical Chemistry, John Wiley and Sons, New York-Chinchester-Brisbane-Toronto-Singapore, 1983, Vol. 3,-p. 397-568; Indicators, E. Bishop (Ed.), Pergamon Press, Oxford, U.K., 1972. These reactions and compounds can be used in the monitoring devices to record exposure history.

Oxidising agents can oxidise reduced dyes and introduce a colour change. Similarly, reducing agents can reduce oxidised dyes and introduce a colour change. For example, ammonium persulfate can oxidise colourless leucocrystal violet to violet coloured crystal violet. Reducing agents such as sodium sulfite can reduce crystal violet to leucocrystal violet. Thus oxidising and reducing agents can be used as indicator reagents. Representative common oxidants (oxidising agents) include: ammonium persulfate, potassium permanganate, potassium dichromate, potassium chlorate, potassium bromate, potassium iodate, sodium hypochlorite, nitric acid, chlorine, bromine, iodine, cerium(IV) sulfate, iron(III) chloride, hydrogen peroxide, manganese dioxide, sodium bismuthate, sodium peroxide, and oxygen. Representative common reducing agents include: Sodium sulfite, sodium arsenate, sodium thiosulfate, sulphurous acid, sodium thiosulphate, hydrogen sulfide, hydrogen iodide, stannous chloride, certain metals e.g. zinc, hydrogen, ferrous(II) sulfate or any iron(II) salt, titanium(II) sulphate, tin(II) chloride and oxalic acid.

Acid-base reactions are colourless, but can be monitored with pH sensitive dyes. For example, bromophenol blue when exposed to a base such as sodium hydroxide turns blue. When blue-coloured bromophenol blue is exposed to acids such as acetic acid it will undergo a series of colour changes such as blue to green to green-yellow to yellow. Thus, acids and bases can be used in conjunction with pH dependent dyes as indicators systems. The following are representative examples of dyes that can be used for detection of bases: Acid Blue 92; Acid Red 1, Acid Red 88, Acid Red 151, Alizarin yellow R, Alizarin red %, Acid violet 7, Azure A, Brilliant yellow, Brilliant Green, Brilliant Blue G, Bromocresol purple, Bromo thymol blue, Cresol Red, m-Cresol Purple, o-cresolphthalein complexone, o-Cresolphthalein, Curcumin, Crystal Violet, 1,5 Diphenylcarbazide, Ethyl Red, Ethyl violet, Fast Black K-salt, Indigocarmine, Malachite green base, Malachite green hydrochloride, Malachite green oxalate, Methyl green, Methyl Violet (base), Methylthymol blue, Murexide, Naphtholphthalein, Neutral Red, Nile Blue, alpha-Naphthol-benzein, Pyrocatechol Violet, 4-Phenylazophenol, 1(2Pyridyl-azo)-2-naphthol, 4(2-Pyridylazo) resorcinol Na salt, auinizarin, Quinalidine Red, Thymol Blue, Tetrabromophenol blue, Thionin and Xylenol Orange.

The following are representative examples of dyes that can be used for detection of acids: Acridine orange, Bromocresol green Na salt, Bromocresol purple Na salt, Bromophenol blue Na salt, Congo Red, Cresol Red, Chrysophenine, Chlorophenol Red, 2,6-dichloroindophenol Na salt, Eosin Bluish, Erythrosin B, Malachite green base, Malachite green hydrochloride, Methyl violet base, Murexide, Metanil yellow, Methyl Orange, Methyl violet base, Murexide, Metanil yellow, Methyl Orange, methyl Red Sodium salt, Naphtho-chrome green, Naphthol Green base, Phenol Red,4-Phenylazo-aniline, Rose Bengal, Resazurin and 2,2′4,4′,4″-Pentamethoxytriphenylmethanol.

Organic chemicals can be detected by the presence of their functional groups. Organic functional group tests are well known and have been developed for the detection of most organic functional groups, and can be used as the basis for the indicator-activator combination. For example, eerie nitrate undergoes a yellow to red colour change when it reacts with an organic compound having aliphatic alcohol (—OH) as functional group. Organic compounds having one or more of the following representative functional groups can be used in the device as activators; alcohols, aldehydes, allyl compounds, amides, amines1 amino acids, anydrides, azo compounds, carbonyl compounds, carboxylic acids, esters, ethoxy, hydrazines, hydroxamic acids1 imides, ketones, nitrates, nitro compounds, oximes, phenols, phenol esters, sulfinic acids, sulfonamides, sulfones, sulfonic acids, and thiols. There are thousands of compounds under each functional group class listed above. For example, the following is a representative list of aminoacids that can be used as activators in the device: alanine, arginine, aspartic acid, cysteine, glutamic acid, glycine, histidine, hydroxylysine, lysine, methionine, phenylalanine, serine, tryptophan, tyrosine, alpha-aminoadipic acid, alpha, gamma-diaminobutyric acid, ornithine and sarcosine. All alpha-amino acids undergo a colourless to purple-violet colour when reacted with ninhydrin. In addition, the following are some specific amino acid tests: 1) Diazonium salts couple with aromatic rings of tyrosine and histidine residues to produce coloured compounds. 2) Dimethylaminobenzaldehyde condenses with the indole ring of tryptophan under acid conditions to form coloured products. 3) alpha Naphthol and hypochlorite react with guanidine functions (arginine) to give red products. The following is a representative list of alpha-amino acids that can be used as solid amines: Lysine, hydroxylysine, alpha, gamma-diaminobutyric acid and ornithine. The following are some further selected examples of organic compounds that undergo a colour change in the presence of a functional group test reagent: Primary, secondary and tertiary aliphatic and aromatic amino bases can be detected with 2,4-dinitro chlorobenzene. The observed colour change is from colourless to yellow-brown. Aliphatic amines, primary aromatic amines, secondary aromatic amines and amino acids react with furfural in glacial acetic acid to give violet Schiff bases. A variety of triphenylmethane dyes react with sulfurous acid to produce a colourless leucosulfonic acid derivative. When this derivative is allowed to react with an aliphatic or aromatic aldehyde, coloured products are obtained. Fuchsin, decolourised with sulfite when exposed to aliphatic and aromatic aldehydes, gives a violet blue colour. Malachite green, decolourised with sulfite when exposed to aliphatic and aromatic aldehydes, gives a green colour.

A large number of reactions are associated with a change in fluorescence rather than a colour change in the visible region. Several fluorescent indicators are known (Vogel's Textbook of Quantitative Inorganic Analysis, Fourth Edition, Longman, p. 776.).

The device and its modifications are not limited to chemical, indicator combinations, which are associated with chemical reactions for producing a colour change. Also included are any two or more compounds, which can undergo a noticeable or measurable physical change, which can be monitored by appropriate analytical equipment. Such changes include particle size, transparency, electric conductivity, magnetism and dissolution. For example, a change in conductivity can be monitored by an electrometer.” (WO9209870).

A range of measurement and communication combinations possible with passive sensing-indicators in the present invention is articulated in Table 1.

TABLE 1
Range or metering possibilities

| Measure | Measurement | Visual | |
|---|---|---|---|
| monitored | taken | communication | Electronic communication |
| Prevailing | Exposure as | Visual reading | Instrument reading of a sensor's |
| level of an | one-dimensional | by a sensor | colorimetry as wavelength, frequency, |
| analyte | diffusion | showing | reflectance, uminescence, fluorescence, |
| OR- | comprising a | moving colour | or quanta of light reflected over space in |
| Cumulative | moving colour- | change | a field of view |
| exposure | band along a | | resulting from scavenging and reaction |
| (exposure | linear strip | | with an analyte in a moving band, and |
| history) | | | passed to the observer by electrical |
| | | | current, potential difference or |
| | | | resistance; potentially communicated by |
| | | | radio signal from remote location to a |
| | | | centre of coordination and relayed |
| | | | further by telecommunications. |
| | | | Instrument reading of the changed |
| | | | electrical conductance, resistance, or |
| | | | potential difference within a printed |
| | | | circuit due to a changed electrical |
| | | | property of a sensor that scavenges and |
| | | | reacts with changing levels of target |
| | | | molecules in a moving reaction front, |
| | | | potentially communicated by radio |
| | | | signal from remote location to a centre |
| | | | of coordination and relayed further by |
| | | | telecommunications. |
| | Exposure as planar | Visual reading by a | |
| | diffusion comprising | sensor showing moving | |
| | an expanding or | colour change | |
| | contracting concentric | | |
| | colour-ring | | |
| | Exposure as an | Visual reading by a | |
| | increasing non-linear | sensor showing moving | |
| | measure into a 3- | colour change | |
| | dimensional shape such | | |
| | as a wedge | | |

The use of the appearance or disappearance of colour, as can be obtained with phenolphthalein composition in the indicator, is a favoured method, as there is no wavelength change as the reaction proceeds, but an absorbance change occurs, which provides greater accuracy in visual detection and interpretation of the progress in metering.

In Table 1 it can be seen that the prevailing level of an analyte or the cumulative exposure to an analyte can be monitored and reported with an automated and passive device according to the present invention. It is also possible to combine both applications into the one device in order to report both prevailing and cumulative levels simultaneously.

In the present invention, prevailing concentrations and cumulative exposure to acid-base, or oxidation-reduction reactants or products are metered in six ways.

In the first, the saturation of colour intensity according to Beer's Law is used to meter levels, by relating colour intensity to the concentration of reaction products formed in the sensing-indicator. This may be undertaken with the ability of the naked eye to discriminate between the development of colour intensity as the analyte progressively diffuses as a migration front into the sensing-indicator and the consequent reaction proceeds. The resulting colour intensity is proportional to the concentration of a prevailing molecule, or mass of reaction products in the case of cumulative exposure, and hence the exposure history.

This form of the present invention is best viewed in the same plane as the migration of the reaction front into deeper layers of reagents, and may involve an instrument capable of measuring the strength of signal or wave length or frequency, from colorimetry, reflectance, luminescence or fluorescence.

In the second, the rate of reaction according to Fick's law is used to meter levels by relating the level of the analyte to the rate of colour movement and/or distance of colour movement along a reaction front established by the special architecture of the sensing-indicator device, that confines the diffusion in a line or a plane. This form of the present invention is best viewed in the perpendicular plane to the migration of the reaction front.

To illustrate the second form, if the substance(s) of a detector film is sealed over its upper and lower surfaces by a barrier film, with its edges exposed, the access of an active reagent, can be restricted to the edges of a laminate. A colour fringe moves from the exposed edge or area, the distance of colour migration being proportional to the time squared in accordance with Fick's Law. Thus if 1 mm of colour migration is apparent in one day, 1.4 mm will appear in two days, under exposure of a constant concentration of target molecules. The same indicator film only needs to be calibrated once for any particular application.

A sensing-indicator of the second from can alternatively be obtained by sealing all edges of a thin disc of the sensing-indicator described above, but now sealed at the edge, and later puncturing its middle so that the migration of colour change is from the centre to the edge. A similar effect for a linear colour migration can be created by sealing an elongated linear strip and exposing one end to an analyte. This second form of the present invention is illustrative of metering along a continuous scale for visual readings by persons untrained in the intricacies of elaborate instruments, for example handlers of perishable food being monitored during storage, transport, distribution, sale and consumption.

In a third form of the invention, indication of a change in the electrical conductance, potential difference, or resistance of the sensor of the present invention can be detected. When powered by a detached power source, such as a battery or solar cell, the electrical reading may be conveyed by radio frequency identification devices now available as printed circuitry on food packages. The signal can be communicated by a transponder of radio signals to a remote centre. There are technologies available in industry for such communication. Inclusive amongst these are Radio Frequency Identification (RFID) tags for packages during distribution, and GSM-based General Packet Radio Service (GPRS); and a description of a container sensor unit that takes readings of temperature and reports them to a base station unit on board a ship for relay by satellite link for viewing over the internet by interested parties is provided by Morris et al. (2003). Whereas these commonly report temperature measured by a thermister sensor, the migrating reaction-front sensor of the present invention can be similarly linked with such circuitry.

Spaces such as food packages, a flowing stream of air or water, air within a room, a volume of water for treatment, or fumigant in a carton of produce are confined to some degree and a certain concentration of target molecules establishes within these environments. Applications of the present invention to report current status will generally involve reporting rising or falling concentrations of a target molecule within such confined spaces.

The level of carbon dioxide within fresh produce packages is reported on a discrete scale with a plurality of individual sensors in patent EP0627363. The objective of the present invention, in contrast, is to adapt one sensor to generate multiple readings.

A meter can be manufactured that reports the prevailing level of the target molecules in an environment by using reversible reactions, such as mixing a buffer with an indicator and a calibrating reagent in an indicating medium.

In the present invention of a moving reaction-front, a rapid response to environmental change is obtained by ensuring a high degree of permeability in the device to forward and backward diffusion of target molecules along a column or a plane, as reactants inputted into or products evolved from, a chemical reaction of dynamic equilibrium within the sensing medium. This way a rapid adjustment is achieved to the new level Within the instrument in response to small changes in the concentration of target molecules in the outside environment, and is reported in a timely manner. The effect may be obtained by the use of a capillary-tube like environment and limited filling of a tube with material to create tortuosity.

High permeability in the indicator medium may be achieved selecting permeable materials for indicator composition and by abutting porous micro-spheres of high volume to mass ratio as an indicating medium in the confines of an elongated vessel; or manufacturing an indicator medium using crystalisation, plasticisation, perforation, polymer expansion, or other means known in the polymer-manufacturing industry to produce enhanced permeability or porosity.

A first method to enhance the sensitivity of the device in detecting small pH changes to an analyte, pH buffers may be used. The buffers should desirably have a pK value close to the pK range of the typified environment being measured and produce a substantial colour change in response to very small changes in the analyte. To illustrate with carbon dioxide metering, enhanced sensitivity may be achieved by the use of amino acids or borate as buffers. The carboamino reaction may be adjusted with combinations of amino acid reactants like lysine or glycine, with or without borate. Desirably, pH buffers should have a pK value close to the pK range of the typified environment being measured and produce a substantial colour change in response to very small changes in hydrogen concentration. Similar methods may be used to measure small changes in oxidation status with, for example, oxygen metering or other gases or liquids of interest.

A second method uses the scavenging action of an indicator to enhance sensitivity of the metering device. When low prevailing levels of a targeted chemical ion are measured, the response to a sensor based upon reversible reactions can be poor, as the low level is beyond the sensitivity range of the instrument. By scavenging low levels of target molecules into a sensor that accumulates molecules in an additive manner, detectable readings may be exhibited in a colour-changing trend.

The form of the invention that reports cumulative exposure can be manufactured with reagents that are either relatively semi-stable or stable at normal operating temperatures. A recharge capability can be obtained for the device if reagents are chosen that will form semi-stable reaction products within an operating temperature range of approximately 0-60° C., but will reverse within a temperature range of approximately 60-80° C. that can be imposed on the device to reverse the reaction by mild heating to recharge it back to the zero value. One such reagent, which fulfils this requirement, is potassium carbonate, a reagent that can be used to measure exposure to acid vapours.

A related application can be applied to the problem with alkaline scavenging reagents used to measure exposure to acidic analytes during manufacture and storage, as they are reactive with carbon dioxide present in the atmosphere, and may be triggered to work prematurely. During manufacture of polymer packaging films, it is desirable to purge carbon dioxide absorbed during storage and handling with mild heating for example by passing film through an oven environment. The reporting device may be commissioned by mild heating to approximately 60-80° C. prior to packing the product, to bring the reported measurement back to zero or close to it.

In accordance with this inventive principle, reversibility in metering alkaline exposure may be achieved by heating acidic scavenging reagents such as acetic and tartaric acid, although the temperature range to achieve a reversal may differ.

In application, the recharge capability may be utilized in the manufacture of a rechargeable instrument to measure exposure to target molecules. The instrument could be re-charged by heating it at temperatures above room temperature, but below a temperature which will detrimentally affect the chemical composition of the reagents or the melting point of materials used in its manufacture.

In the management of quality, consumers wish to obtain the freshest of supplied stocks, whilst distributors wish to market stocks with some deterioration in quality up to the point of consumer acceptability. Thus, some conflict exists between the interests of customer and supplier over freshness of deteriorating food or other biological products.

In the present invention, the metering can be achieved by deployments that target communications at different audiences, wherein some interested parties are alerted in an early-warning, when the level of exposure is low, whilst others in a disparate class of recipients receive the communication when the reaction has progressed to an advanced stage, when the level of exposure is higher.

This may combine various modes of metering disclosed in the following section on colour possibilities. The coded message may be received by food-supply staff or quality-control staff in the trade using special instrumentation, such as a bar-code scanner and take the form of a missing or additional bar-code using indicators that appear or disappear. A measurement may also be taken by an instrument, such as colour intensity or the quantum of colour scanned over a given space.

The form of electronic communication, coded to a particular recipient class such as stock clerks, may include the bar-code readings obtained by reflectance.

Indicators can be mixed to provide an expanded spectrum of colour change to choose from, for example changes from acid to neutral and onto alkaline environments are widely reported in chemical technology with universal indicator. The resulting colour changes can be correlated with varying levels of exposure to achieve a scale.

One method according to the present invention, to convert a single colour indicator to another, for example from pink to black, as with an application where an electronic barcode scanning is required in the distribution of perishable, packaged chopped and diced vegetables' to a retail store, is to contrast it against a green coloured transparent layer placed above or green coloured background material below it. Upon exposure, if the colour change in the indicator is from pink to colour-less, then the effect of the green contrast layer is to alter the colour change to one where black turns to green.

Alternatively, the indicator may be mixed with a colouring reagent that does not participate in the exposure reaction, which will convert the colour change into one more desirable for communication purposes.

Many chemical reactions that result in an indicator changing colour depend upon the presence of water for colour change to occur; this dependence can involve the processes of migration of the target molecules into the indicating medium, solubilisation and ionization. Efficacious indicating materials therefore are selected for affinity with water for such applications and a humectant may be mixed with the sensing-indicator. A problem exists under humid operating conditions, as moisture uptake can cause the reaction front to be dissipated and the measure to be lost. This effect can be controlled by either adjusting the concentration of the humectant, or establishing a selective permeation of the target molecules through an encasing material like silicone or polyethylene which will limit moisture migration into the sensing-indicator, or by selecting plasticisers for indicator composition that prevent excessive moisture uptake, or by deploying with the indicator various salts that are known to regulate humidity within a particular range, or a combination of these methods.

It is possible that the invention could be used to measure acid or alkaline analytes, or oxidation or reduction analytes.

Packaged food are sensitive materials to ionic disturbance, and ionic leakage and migration into the sensing material through the wall of the package is to be avoided, otherwise quality and safety may be impaired. Selective transmission of non-ionic molecules would be advantageous, and this can be achieved by a separation layer that is selective in transmission, for example it may be composed of a silane like silicone that transmits only non-charged molecules like carbon dioxide.

Another method is to select a polymer layer as a membrane between the sensitive storage product and the sensor with micropores of diameters sufficiently narrow to permit diffusion of smaller target molecules, whilst excluding larger non-target molecules.

Still another method is to use filtering layers or scrubbers to remove confusing molecules from the sampling stream between the generating source and the indicating device. An example is where molecules are present of confusing, opposing chemical species to the crude measures of pH or oxidation state. An illustration is where volatile bases present in degrading fish are present in a fish package whilst carbon dioxide evolved by decomposing bacteria is being measured with an alkali mixed with an indicator. Deployment of filtering layers or scrubbers should remove confusing molecules of the degrading proteins and amines from the food package. Alternatively, the carbon dioxide evolved from the metabolism of bacteria, an acid vapour, could be scrubbed so that amine formation, alkaline in reaction, could be measured more accurately.

To relate readings to prevailing concentrations or cumulative exposure, it is important to calibrate the indicator response to exposure. In some industrial applications, exposure to low concentrations for short periods of time will require a high degree of sensitivity, for example where indicators are used to reporting loss of integrity in a package seal with exposure to oxygen or carbon dioxide in the air. To the contrary, for monitoring vehicle emissions over an extended period, a relatively higher exposure history would be of interest.

A method for detection of low prevailing levels is to set a small differential between the indicator and the target level, and to use buffers known in science to resist only a small change in pH, so that minor changes in chemical equilibria will trigger a response in the sensor.

One method to calibrate between high and low exposures, as a method more of coarse rather than fine tuning, is by metering a proportion of the molecules generated by a chemical process, rather than all molecules. This can be achieved by restricting access to the sensing-indicator by narrowing access pores or creating tortuous access routes in apertures between the source of generation of the target molecules and the sensing-indicator device.

Variable permeability of the sensing-indicator material and/or that of encasing material such as bather film or over the aperture of an intake device, can be similarly used to calibrate response to exposure, and among possible methods to vary permeability are material selection, varying plasticiser composition or the degree of crystalisation in manufacture. Perforations can also be used to increase the surface area exposed to target molecules, relative to the volume of indicator, to accentuate colour change in certain regions of the indicator and so refine interpretations of the level of exposure attained. The size of a single aperture at the intake of device can also be used to calibrate the rate of diffusion.

In the cumulative exposure form, a film for wide application can be prepared by manufacturing an indicator with a thickness of sufficient magnitude to scavenge a wide number of molecules, from few to many, so that an interpretation chart for each application provides the interpretation pertinent to the given application. This is achieved by virtue of the independence that the diffusion rate has of the concentration gradient.

Another calibration method is to vary the reaction rate with buffers, whilst another alternative is to deploy varying doses of reagent and indicator, and to vary the reagent / indicator ratio, that will react with the target molecules until the desired equilibrium is reached and colour change will occur.

Still another, is to vary the thickness of the indicator to alter the effect of the reaction, on change in the indicator as visible colour observed by the naked eye, or as colour measured by an electronic instrument. With increasing thickness of the indicator material, whether disposed in a tube or a film, progressive migration of target molecules through successive layers results in a migration of the reaction front toward un-reacted colour reagent. When viewed at the perpendicular to a film indicator, increasing thickness will enhance the sensitivity of the exposure-indicating meter as a useful instrument to higher exposures, since the colour intensity will be lost at a slower rate with increasing exposure. When viewed in the same plane as the migration front, as in a tubular disposition of the device, providing an interpretation as a band-reading like that provided by a conventional thermometer, the longer the tube or strip of film, the greater the scale provided for metering exposure.

The rate of migration of the reaction front, the velocity, can be used as a calibration method for interpretation purposes with application of the time dimension. The rate of progress in the development or loss of colour intensity as the front moves away from the observation post at an angle of 90° into deeper layers of the indicator can be used as a calibration method. Alternatively, calibration may be obtained from the rate of linear migration of a colour-band in the same plane as the observation post of linear colour-band devices, or radial migration in the case of colour-ring devices.

The extent of migration of the reaction front, a measure of distance can also be used to meter exposure and obtain calibration against levels of exposure.

In the case of electrical measurement of changes in the scavenging sensor, the gain or loss in time of an electrical property such as current or resistance, due to the migration of the reaction front, may be calibrated with changes in the surrounding environment.

These calibration methods can be used solely or in combination to meter exposure to target molecules.

As outlined above, there are two types of scale that the cumulative exposure indicator can be measured by, a discrete and a continuous one.

One form is the progressive exposure and reaction of target molecules with a reagent to form products in a continuous scale to indicate the degree of deterioration in quality, and again calibration of the device is important.

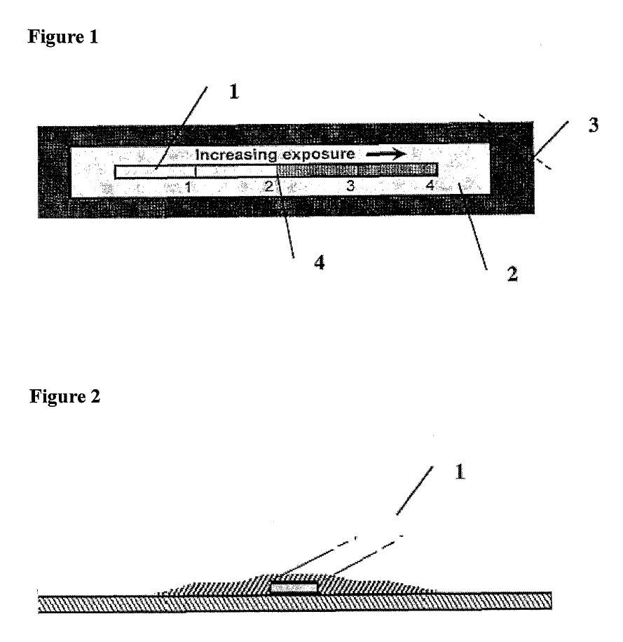

Metering can be communicated in a continuous scale by confining diffusion of the reaction in one dimension, and can be calibrated according to exposure by adjusting the velocity of the reaction front according to the methods disclosed in this invention. One such method confines one-dimensional diffusion in an elongated vessel, permeable or porous at one end, as shown in FIG. 1. Referring to FIG. 1, it can be seen that a strip of printed indicator, or indicator film, or fluid-filled cylinder with indicator gel is disposed linearly (1) and is covered by a barrier layer (2) to confine diffusion in one dimension. The one-dimensional progression communicates metered exposure visually, reflectantly, luminescently, fluorescently, or by other radiation technology. The device is commissioned by removal of a sealing layer (3), for example with scissors or peeling away a barrier film or puncturing action or releasing a blister or any means known in the packaging industry to remove a seal, and a linear or non-linear scale printed along the linear progression in colour (4), provides a reading and facilitates interpretation. The figure shows linear progression in colour change to Level 2 out of 4 levels on the scale as a result of exposure.

FIG. 2 shows a view in section to illustrate how the diffusion is confined linearly in space with a narrow film sealed with encasing material, in this form by two laminates, which may similarly be achieved with tubes filled with gel indicator.

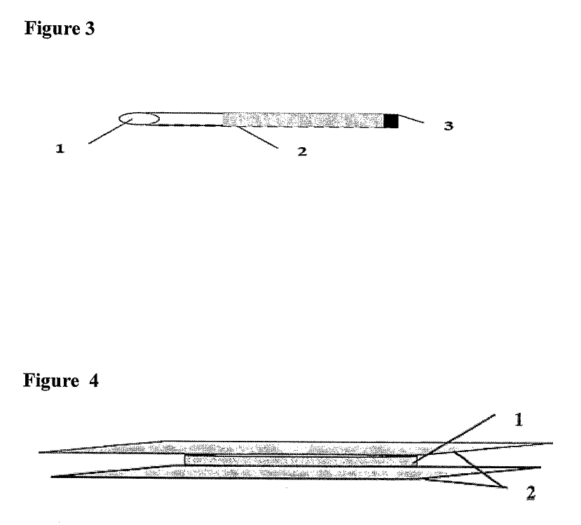

The device can be made in the form of a dip-stick instrument for submergence in liquids, possibly with a floatation ring to orient it vertically, to meter exposure from concentrations of analytes in solution, as shown in FIG. 3. Referring to FIG. 3, it can be seen that a solvent-proof protective tip chosen for selective permeation of analyte (1) permits diffusion of the analyte into the measuring tube, then progressive reaction with the reagent and indicator under diffusion migrates the colour front in response to exposure along the tube, interpreted using a printed scale for readings (2), whilst an impermeable seal is maintained at the opposite end of the tube (3).

A second method uses planar diffusion in two dimensions from the edge of a film toward the centre, as shown in FIG. 4. Referring to FIG. 4, it can be seen that a disc of indicator print or film (1), is covered by barrier layers like a sandwich, (2) to confine diffusion in a plane migrating from the edge toward the centre, and the progression communicates metered exposure visually, luminescently, or fluorescently.

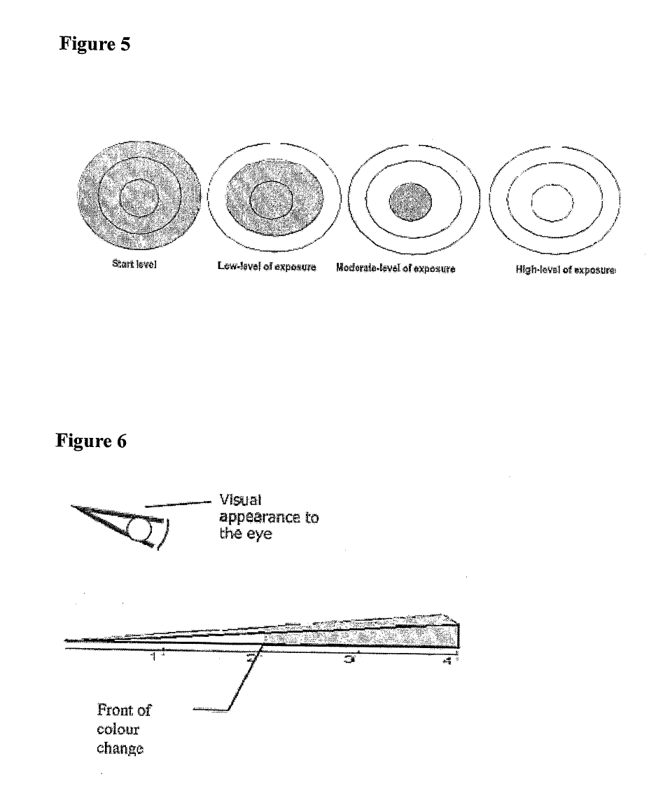

An aerial view is illustrated in FIG. 5 of the disc form that applied planar migration during operation. Referring to FIG. 5, it can be seen that a linear or non-linear scale is printed as concentric circles along the radial progression in colour onto the upper sealing layer. Colour migrates in this form from the edge towards the centre, because an edging seal is broken and exposure drives the reaction toward the centre. Colour change at each concentric circle represents an increasing level of exposure according to a scale of interpretation calibrated for the particular industrial application. In FIG. 6, it can be seen that colour changes from coloured to colour-less with increasing exposure, from the edge toward the centre. It can be seen that exposure to target molecules has moved the colour change from the outer edge toward the centre by one level on the printed scale.

The device can alternatively be sealed and a hole punched in its middle for the migration of colour change to radiate from a central position.

FIG. 6 shows a third form that shapes the indicator into the tapered form of a wedge, pyramid, cone or other three dimensional shape so that colour change will progress with increasing exposure from the fine tip to the thick base. Referring to FIG. 6, it can be seen that exposure has moved the front of the colour change, from the thin end of the wedge toward the thick base, to level 2 on the interpretation scale.

The progression of colour-band migration in the above embodiments can be made to communicate metered exposure visually, luminescently, or fluorescently.

One method to achieve an acceleration or deceleration whilst the colour band migrates on its journey from the intake position to the terminus, is to provide a further port of entry to the analyte at stations along the line in addition to the intake aperture. This may be achieved at stations along the line of colour migration by reducing the thickness of barrier film at that section of line, or the layers of barrier film, or the permeability of barrier film, including perforations or incisions made though the barrier film. Another is to join various separate lines of indicator into a continuous one; the composition of each section may vary in respect of permeability, doses of reagent, selection of buffer or levels of buffering.

In some industrial applications, a combination of readings in continuous and discrete scales may be required. An example of the use of coded communications directed at disparate parties is the distribution chain for food to indicate the degree of exposure from increasing deterioration in quality of food. This can be achieved by a special adaptation of the moving colour-band device to modify the continuous scale into a graduated scale.

The moving colour band can be modified to produce a graduated scale by the use of masking over sections of the line of moving colour band or the printing of alpha-numeric text or symbols under the band of indicator. The objective is to progressively mask or reveal colour change along a line of colour diffusion.

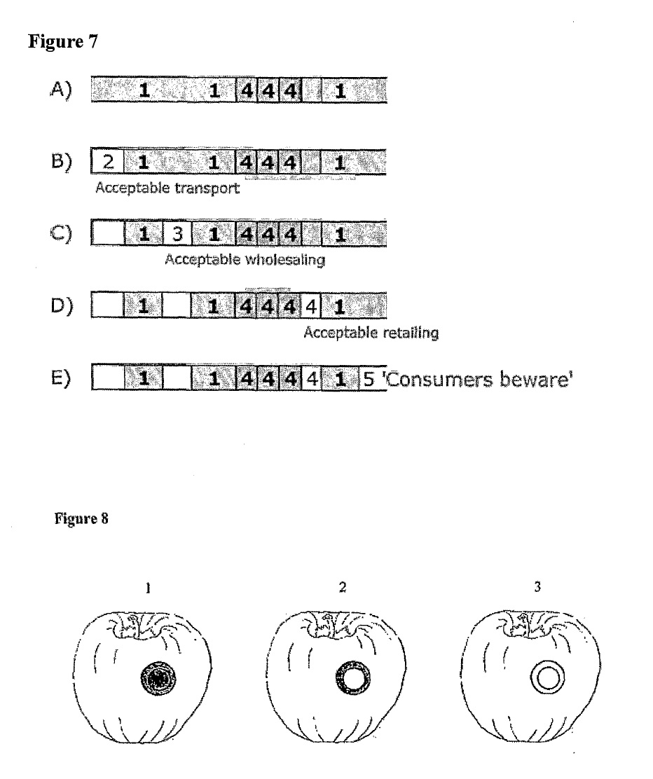

By way of example, a continuous scale of the moving colour-band is made to produce a graduated scale and codified reports to various parties in the distribution of food about the level of freshness. In FIG. 7 it is shown how this can be achieved, and in this illustration, the moving colour band migrates from left to right. The device uses purple masking as a layer in sections over the purple colour band below. If an analogy is drawn with a rail-train underground subway, then as the colour-band migrates along the line, it becomes visible like a rail car at stations along a subway.

In another adaptation, if the band of purple indicator overlies purple print below as a ceiling colour and the colour change migrates linearly, then the purple print below will be unveiled by the passing reaction front which turns colourless and the underlying print is made visible to the observer.

This application modifies the continuous scale of the moving colour-band to produce a graduated scale and codified reports to various parties in the distribution of food about the level of food spoilage. In FIG. 7, it can be seen that the moving colour band migrates from left to right. The device uses masking layers, in some applications there are layers over the moving colour band, in others the band of indicator overlies coloured print below. Stages A to E in the progression of the colour band are shown.

Area 1 is a colour print that masks the progression of the progression of the front of colour change from the observer, the colour change occurs beneath these panels, which overlay the indicator below.

At stage A—The migration of the reaction front whilst under manufacture inventory has caused no discernible product deterioration

At Stage B—The migration of the reaction front whilst under transport of product from manufacturer to wholesaler has consumed the tolerable change in the indicator, causing the Area 2 to change colour from pink to transparent

At Stage C—The migration of the reaction front whilst under wholesaling of the product has consumed the tolerable change in the indicator, causing the Area 3 to change colour from pink to transparent

At Stage D—The migration of the reaction front, whilst under retailing of the product, has consumed the tolerable change in the indicator, causing the Area 4, one of the 4 bar-codes, to change colour from pink to transparent, communicating a coded message interpretable only by retail staff, whilst consumers are oblivious to the condition

At Stage E—Area 5 comprises is a coloured masking layer of the indicator overlaying a printed message in ink of the same colour of the indicator. As the reaction front migrates, the colour of the indicator changes from pink to colour-less, and the masking layer disappears, revealing a universal message printed in pink and previously blanketed underneath the formerly pink and now transparent colour band, advising consumers in text and or symbol that the product is unfit for purpose.

FIG. 8 shows a sticker form of the present invention placed on the exterior surface of a piece of fruit undergoing ripening/senescence. In this case, the device is punctured at its centre and with accumulated respiration and cumulative exposure to carbon dioxide evolution from respiration or ethylene exposure from ripening process, the metering device shows progressive measures at levels 1 through to 3 from a colour ring that expands as the reaction front enlarges. The device could similarly be disposed on the interior surface of permeable food packaging, or the interior surface of impermeable food packaging, for example wrapped food like meats and fish, or as a gasket in the screw-cap of a milk container.

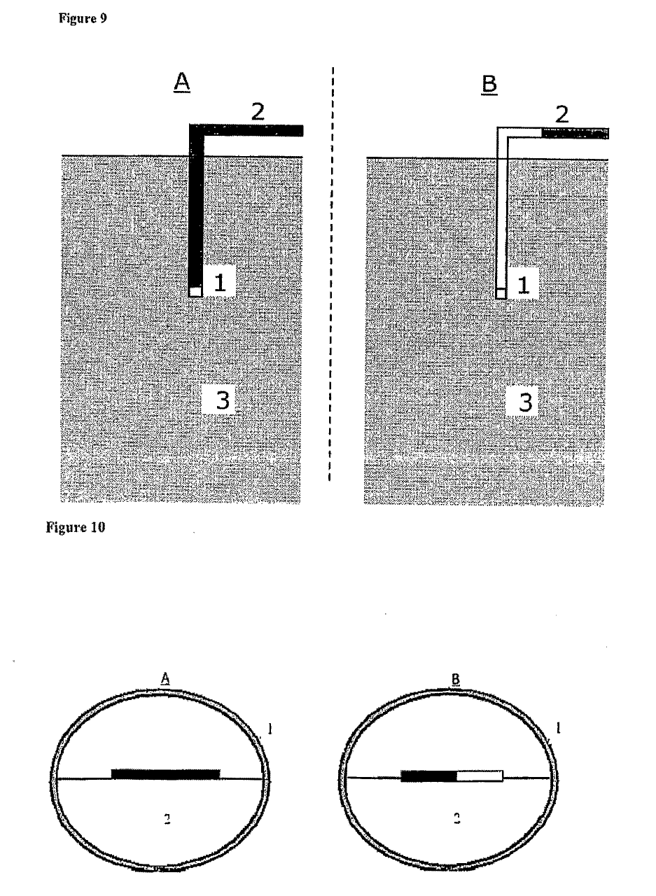

FIG. 9 shows the form of the invention shown in FIG. 3 configured to be deployed as a device for monitoring gas levels in soil, such as carbon dioxide from the metabolism of soil organisms. At Stage A in FIG. 9, the device is deployed, whilst at Stage B the cumulative carbon dioxide scavenged from the soil has moved the colour band along the soil surface to a level in given time that is commensurate with an active population of soil microbes. In FIG. 9, the sealing cap 1 is water proofed but is permeable to carbon dioxide, the barrel marked 2, angled at 90 degrees to the probe section, is graduated to establish a scale, and the soil profile 3, is shown in section.

FIG. 10 shows the form of the invention configured to be disposed in the exhaust stream of a motor vehicle. In FIG. 10, the tail pipe 1 is observed from behind the vehicle as a government regulator might do from a vehicle travelling behind the polluting vehicle. The exposure device is shown freshly deployed at Stage A, and at half the scale on the colour-band 2, at Stage B. If the pollution limit under a license is the length of the colour band in FIG. 10, then the owner of the vehicle and the government enforcer can conclude that 50 percent of the permissible emissions have been discharged and by deduction, 50 percent of the current license is left.

### REFERENCES

Bower, J. H. (2001). The relationship between respiration rate and storage life of fresh produce. PhD thesis, Centre of Horticulture and Plant Sciences, University of Western Sydney, Hawkesbury Campus.
Brash, D. W., Charles, C. M., Wright, S. and Byrcroft, B. L., 1995, Shelf life of stored asparagus is strongly related to postharvest respiratory activity. Postharvest Biology and Technology 5 77-81
Morris, S. C., Jobling, J. J., Tanner, D. J. and M. Forbes-Smith (2003). Predication of Shelf-life for Fresh Produce Transported by Refrigerated Containers. Acta Horticulturae. 604(1), pp. 305-311.
Riva, M. (1997) Time-temperature indicators, a review by Marco Riva, University degli Studi di Milano, Italy 1997

1-21. (canceled)

22. An indicator system for determining and indicating a prevailing concentration or exposure history of an analyte, comprising:
a carrier medium provided with one or more reagents able to react with the analyte and produce a visible and diffusing reaction front;
at least one barrier layer to confine diffusion of the analyte within the carrier medium;
at least one aperture to allow intake of the analyte into the carrier medium to be able to react with the one or more reagents; and,
a readable scale to provide a visible indication of a location of the visible reaction front.

23. The indicator system as claimed in claim 22, wherein the indicator system comprises a first barrier layer and a second barrier layer, whereby the carrier medium is disposed between the first barrier layer and the second barrier layer.

24. The indicator system as claimed in claim 22, wherein at least part of the at least one barrier layer is transparent or translucent to an observer.

25. The indicator system as claimed in claim 24, wherein the readable scale is a graduated scale provided on the at least one barrier layer.

26. The indicator system as claimed in claim 24, wherein the carrier medium and the at least one barrier layer are substantially circular in shape or elongated as a column.

27. The indicator system as claimed in claim 25, wherein the graduated scale is formed of concentric rings or as linear units.

28. The indicator system as claimed in claim 23, wherein the at least one aperture is an open ring or rectangle formed between the first barrier layer and the second barrier layer.

29. The indicator system as claimed in claim 23, wherein the at least one aperture is at least one hole formed in the first barrier layer or the second barrier layer, and the perimeter of the first barrier layer and the second barrier layer is sealed.

30. The indicator system as claimed in claim 22, wherein the at least one aperture contains a material permeable to the analyte.

31. The indicator system as claimed in claim 22, wherein the visible and diffusing reaction front is indicated by a change in colour.

32. The indicator system as claimed in claim 22, further comprising a phase transfer agent composed in combination with a pH indicator dye, to make a formed dye ion pairing readily soluble in hydrophobic polymers, and being further buffered by an excess of the phase transfer agent.

33. The indicator system as claimed in claim 22, further including an attachment layer for adhering the indicator system to an object which can produce the analyte.

34. A method of determining and indicating a prevailing concentration or exposure history of an analyte, comprising the steps of:
attaching an indicator system to an analyte producing object, the indicator system including: a carrier medium provided with one or more reagents; at least one barrier layer to confine diffusion of the analyte in or along the carrier medium; at least one aperture to allow intake of the analyte to the carrier medium; and, a graduated scale;
allowing the analyte to react with one or more reagents and produce a visible and diffusing reaction front; and
whereby, the graduated scale is readable to provide a visible indication of the progression of the reaction front.

35. A device for determining and reporting a prevailing concentration or exposure history of an analyte, comprising:
an inert and permeable or porous carrier medium able to host a chemical reaction and provide for controlled diffusion of the analyte, the carrier medium provided with at least one variable property from varying density, porosity, permeability, crystallization, plasticisation, perforation, polymer expansion, or a column of microspheres;
an impermeable barrier material to confine and route diffusion of the analyte along the permeable or porous carrier medium;
one or more reagents loaded into the carrier medium to react with the analyte and to provide an indication of a reaction front using either chemically stable, semi-stable or unstable reactions;
a quantitative scale for measurement of exposure to the analyte, either as graduations along a metric continuum for visual readings of progress of the migrating reaction front generated by diffusion of the analyte, or as a signal associated with a change in an electrochemical or electromagnetic property of the one or more reagents;
an aperture for intake and absorption of the analyte into the device; and,
an attachment means for positioning the device in relation to a source of the analyte, or within a chamber about the source of the analyte.

36. The device as claimed in claim 35, wherein the carrier medium is composed of a water-soluble carbonaceous polymer or a water-insoluble polymer with chemico-physical properties to calibrate the migration of the reaction front,.

37. The device as claimed in claim 35, wherein the carrier medium and barrier material are geometrically configured to calibrate the migration of the reaction front by at least one of the following: a column of micro-spheres, a gel-filled tube, number and length of nanotubes, a strip or disc of film, a strip or disc of film of variable thickness including tapered shape, a strip or disc of film with tortuosity in intake and pathway of diffusion, the size of a single aperture at the intake, the number of aperture intakes, and combined surface area of apertures.

38. The device as claimed in claim 35, wherein the one or more reagents are selected from the group consisting of, titration reagents, oxidation-reduction reagents, precipitation reagents, a diluent, a conjugate, an antigen, and an antibody.

39. The device as claimed in claim 35, further including a window for visually monitoring the progress of the migrating reaction front, the window provided by transparent or translucent materials positioned over the moving reaction front.

40. The device as claimed in claim 35, wherein the scale is a graduated scale having masking in sections over the pathway of the reaction front to either hide or reveal the migrating reaction front at certain positions.

41. The device as claimed in claim 35, wherein the device is provided in a chamber with a specimen and a microbiological growth substrate to detect and meter microbiological populations and their metabolism.
